(19) United States
(12) Patent Application Publication
(10) Pub. No.: US 2025/0143650 A1
(43) Pub. Date: May 8, 2025

(54) PHYSIOLOGICAL METRICS FOR DETERMINING STROKE RISK

(71) Applicants: California Institute of Technology, Pasadena, CA (US); University of Southern California, Los Angeles, CA (US)

(72) Inventors: Yu Xi Huang, South Pasadena, CA (US); Simon Mahler, Pasadena, CA (US); Changhuei Yang, South Pasadena, CA (US); Charles Liu, San Marino, CA (US)

(21) Appl. No.: 18/934,954

(22) Filed: Nov. 1, 2024

Related U.S. Application Data
(60) Provisional application No. 63/547,033, filed on Nov. 2, 2023.

(51) Int. Cl. A61B 5/00; A61B 5/026; G16H 40/67; G16H 50/30

(52) U.S. Cl. CPC A61B 5/7275; A61B 5/0261; A61B 5/4064; A61B 5/6803; A61B 5/7267; G16H 40/67; G16H 50/30

(57) ABSTRACT

Techniques for determining stroke risk are provided. In some embodiments, the techniques may involve causing, using one or more light sources disposed on a headset worn by a user, light to be emitted into the head of the user. The techniques may involve obtaining, using one or more light detectors disposed on the headset, information indicative of light reflected from one more structures within the head, wherein a portion of the obtained information spans a time period during which the user was holding their breath. The techniques may involve based on the obtained information, determining one or more cerebral blood metrics. The techniques may involve providing a representation of the one or more cerebral blood metrics as input to a trained machine learning model, and determining a likelihood the user will experience a stroke over a predetermined future time period based on an output of the trained machine learning model.

### CROSS-REFERENCES TO RELATED APPLICATIONS

This application claims priority to and benefit of U.S. Provisional Patent Application No. 63/547,033, filed on Nov. 2, 2023, which is hereby incorporated by reference in its entirety and for all purposes.

### FEDERALLY SPONSORED RESEARCH OR DEVELOPMENT

This invention was made with government support under Grant No. EY033086 awarded by the National Institutes of Health. The government has certain rights in the invention.

### FIELD

Certain aspects generally pertain to determining stroke risk.

### BACKGROUND

Stroke is a global health concern, with a distressingly high incidence, and substantial morbidity and mortality. Annually, more than ten million people worldwide are impacted by strokes, imposing a heavy toll on affected individuals and their families, with significant health, financial, and quality of life burdens. In the United States alone, strokes affect nearly 800,000 individuals each year. Identifying patients likely to experience stroke may aid in prescribing and/or monitoring lifestyle changes or medications that decrease likelihood of stroke. However, identifying patients likely to experience stroke or determining a patient's stroke risk is difficult or impossible.

### SUMMARY

Techniques disclosed herein may be practiced with a processor-implemented method, a system comprising one or more processors and one or more processor-readable media, and/or one or more non-transitory processor-readable media.

According to some embodiments, a method for determining stoke risk may involve causing, using one or more light sources disposed on a headset worn by a user, light to be emitted into the head of the user. The method may further involve obtaining, using one or more light detectors disposed on the headset, information indicative of light reflected from one more structures within the head of the user, wherein a portion of the obtained information spans a time period during which the user was holding their breath. The method may further involve based on the obtained information, determining one or more cerebral blood metrics. The method may further involve providing a representation of the one or more cerebral blood metrics as input to a trained machine learning model. The method may further involve determining a likelihood the user will experience a stroke over a predetermined future time period based on an output of the trained machine learning model.

According to some embodiments, a system for determining stroke risk may comprise: a headband configured to encircle a head of a wearer of a headset; a plurality of light sources attached to the headband; a plurality of light detectors attached to the headband; and one or more processors. The one or more processors may be configured to: cause, using the one or more light sources, light to be emitted into the head of the wearer; obtain, using the one or more light detectors, information indicative of light reflected from one more structures within the head of the wearer, wherein a portion of the obtained information spans a time period during which the wearer was holding their breath; based on the obtained information, determine one or more cerebral blood metrics; and determine a likelihood the wearer will experience a stroke over a predetermined future time period based on the one or more cerebral blood metrics.

According to some embodiments, a method for determining stroke risk may involve causing, using one or more light sources disposed on a headset worn by a user, light to be emitted into the head of the user. The method may further involve obtaining, using one or more light detectors disposed on the headset, information indicative of light reflected from one more structures within the head of the user, wherein a portion of the obtained information spans a time period during which the user was holding their breath. The method may further involve based on the obtained information, determining a cerebral blood flow as a function of time and a cerebral blood volume as a function of time, wherein the cerebral blood flow and the cerebral blood volume include the time period during which the user was holding their breath and a baseline time period before the user was holding their breath. The method may further involve determining a likelihood the user will experience a stroke over a predetermined future time period based on the cerebral blood flow and the cerebral blood volume.

According to some embodiments, a method of training a machine learning model to predict stroke risk may involve obtaining training data, the training data comprising, for a group of users, representations of cerebral blood metrics, wherein for each training sample, a portion of the obtained data spans a time period during which the user was holding their breath, and wherein each training sample includes a corresponding ground truth stroke risk for the user. The method may further involve providing the training data to a machine learning model, wherein the machine learning model takes, as input, the representations of the cerebral blood metrics and generates, as an output, a prediction of stroke risk. The method may further involve updating the machine learning model based on differences between the ground truth stroke risk and the predicted stroke risk to generate a trained machine learning model configured to predict stroke risk.

These and other features are described in more detail below with reference to the associated drawings.

### BRIEF DESCRIPTION OF THE DRAWINGS

FIG. 1 is a schematic diagram of components of a system that generates stroke likelihood data in accordance with some embodiments.

FIGS. 2A and 2B are diagrams illustrating an example headset in accordance with some embodiments.

FIG. 3 is a diagram illustrating example light paths through the skull and brain of a patient in accordance with some embodiments.

FIG. 4 illustrates graphs depicting example techniques to determine cerebral blood flow in accordance with some embodiments.

FIG. 5 illustrates example cerebral blood volume, cerebral blood flow, and cerebral blood oxygen during a breath holding task in accordance with some embodiments.

FIG. 6 illustrates examples graphs of cerebral blood flow and cerebral blood volume during a breath holding task in accordance with some embodiments.

FIG. 7 is a flowchart of an example process for determining stroke likelihood data in accordance with some embodiments.

FIG. 8 is a flowchart of an example process for training a machine learning model to predict a stroke likelihood in accordance with some embodiments.

FIGS. 9A and 9B depict example experimental data in accordance with some embodiments.

FIG. 10 is a diagram of components of an example computing device in accordance with some embodiments.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

Stroke is a global health concern, with a distressingly high incidence, and substantial morbidity and mortality. Annually, more than ten million people worldwide are impacted by strokes, imposing a heavy toll on affected individuals and their families, with significant health, financial, and quality of life burdens. In the United States alone, strokes affect nearly 800,000 individuals each year. Identifying patients likely to experience stroke may aid in prescribing and/or monitoring lifestyle changes or medications that decrease likelihood of stroke. However, identifying patients likely to experience stroke or determining a patient's stroke risk is difficult or impossible using conventional techniques. In particular, conventional techniques typically utilize a questionnaire that evaluates health factors, such as hypertension, cholesterol levels, diabetes, and atrial fibrillation, etc. along with lifestyle-related factors such as body mass index, exposure to air pollution, smoking, unhealthy diet, alcohol consumption, and low physical activity, and the like. These risk profiles are based on population-level data and may be useful for a general assessment. However, such questionnaires are not definitive for determining the need for invasive or non-invasive evaluations, nor for guiding surgical or pharmacological interventions. For example, such questionnaires may give rise to a stroke risk prediction that is too general, and therefor, inaccurate. Moreover, use of such questionnaires may not accurately and precisely indicate whether various interventions (whether surgical, pharmacological, or behavioral) have modified a given patient's stroke risk, e.g., to determine whether a given intervention has lowered the patient's risk of stroke.

Disclosed herein are techniques for utilizing physiological metrics to predict stroke risk. In particular, the techniques disclosed herein utilize cerebral perfusion metrics, which may include cerebral blood volume (CBV), cerebral blood flow (CBF), and/or cerebral blood oxygenation (CBO), to predict stroke risk. The cerebral perfusion metrics, sometimes referred to herein as “cerebral blood metrics,” may be obtained during a period of time that includes time a patient is holding their breath. Changes in the cerebral blood metrics during the breath-holding time period relative to a baseline time period, and/or changes in the cerebral blood metrics in the time period after the breath-holding time period may be utilized to predict stroke risk during a future time period. Because patients with a relatively high stroke risk have stiffer blood vessels, their blood vessels may be relatively harder to dilate. Accordingly, patients with a relatively high stroke risk may be observed to have relatively larger increases in cerebral blood flow and relatively lower changes in cerebral blood volume relative to patients with a low stroke risk. Other changes, such as slope of change in cerebral blood metrics, timing characteristics of the return to baseline of cerebral blood metrics after breath-holding terminates, etc. may be characterized. In some embodiments, a trained machine learning model may be provided with either extracted features of cerebral blood flow, or with raw cerebral blood flow metrics data, and may generate a stroke prediction risk based on the cerebral blood flow metrics.

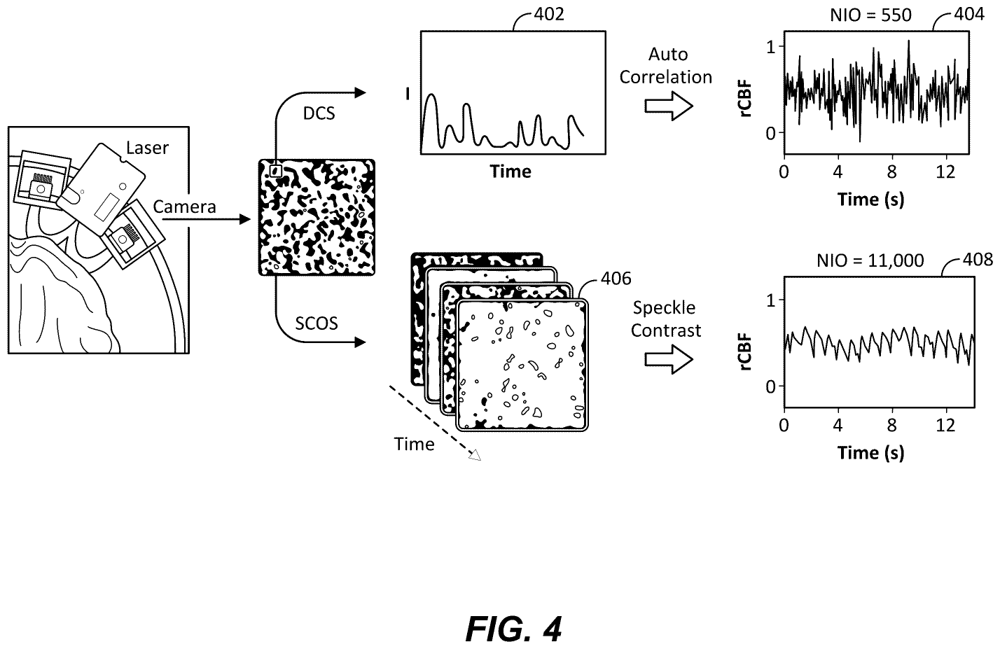

Cerebral blood metrics may be determined using data characterizing light reflected from various structures with a brain of a patient. For example, in some implementations, cerebral blood volume and/or cerebral blood flow metrics may be determined by transmitting light in an infrared wavelength range into the brain of the patient and measuring reflected light. In some implementations, cerebral blood flow may be determined using speckle contrast optical spectroscopy (SCOS), described below in more detail in connection with FIG. 4. In some embodiments, cerebral blood oxygenation may be determined by transmitting light at two wavelengths and measuring reflected/absorbed light at the two wavelengths. The two wavelengths may include an infrared wavelength and a near infrared wavelength.

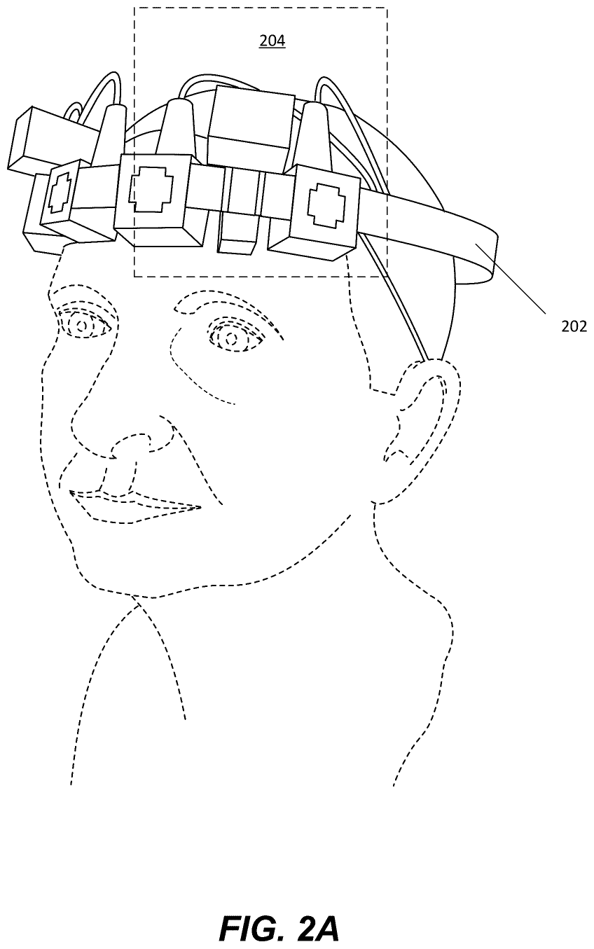

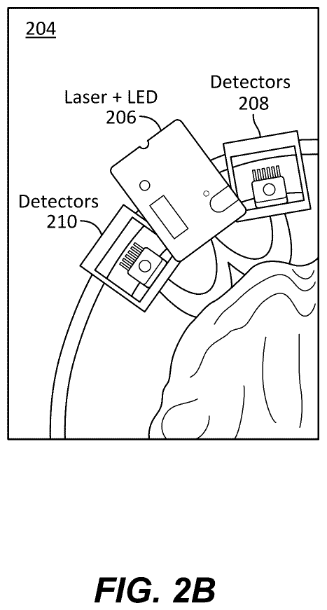

In some implementations, light may be transmitted into the brain of the patient using a light emitter, which may be a laser, a light emitting diode (LED), or the like. Scattered light may be captured using a light detector, which may be a camera, a photodetector, a single-photon avalanche diode (SPAD), a SPAD array, or the like. In instances in which cerebral blood oxygenation metrics are determined and therefore, in which light is transmitted at two wavelengths, two light sources (e.g., a laser and an LED) may be used to transmit light, and two detectors (e.g., a camera and a photodetector) may be used to capture reflected light. In some embodiments, two (or more) light sources may be packaged as one light emission package, and two (or more) detectors may be packaged as one light detection package. In some implementations, light sources and/or light detectors may be disposed on a headband configured to encircle the head of the patient (e.g., around the forehead). In some implementations, light sources and light detectors (or light emission packages and light detection packages) may be affixed to the headband at different locations. For example, the light sources and/or light emission packages may be affixed to the headband at positions corresponding to a forehead of the patient, the parietal lobe of the patient, the frontal lobe of the patient, etc. In some implementations, a light source and light detector may be fiber free in that there is no fiber coupling to either the light source or the light detector. Accordingly, all components of the headset may be head-mounted, which may reduce noise emanating from optical fibers' movement. In some implementations, the laser and the camera/detector may be placed directly atop the user's skin. An example of a headset is shown in and described below in connection with FIGS. 2A and 2B.

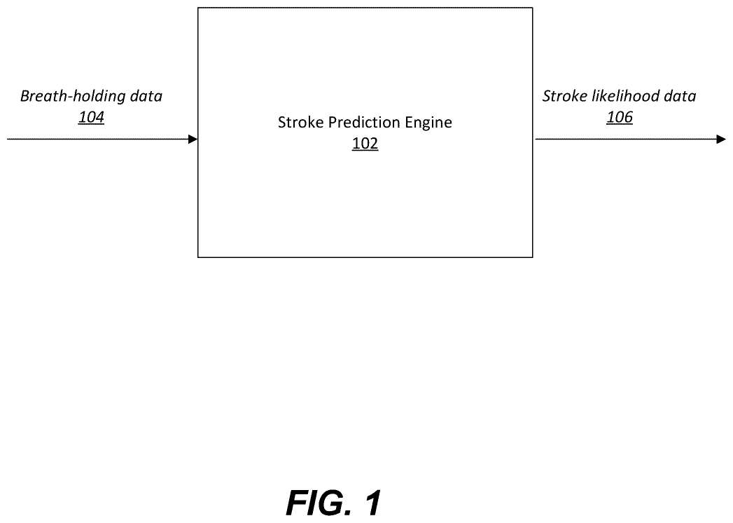

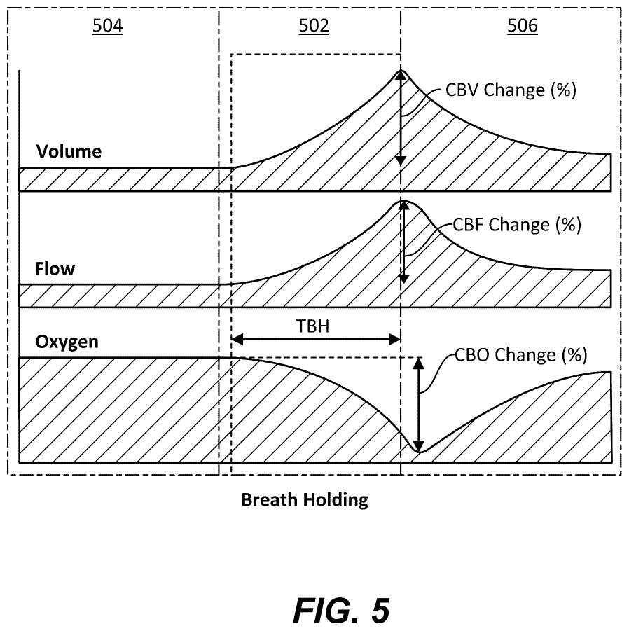

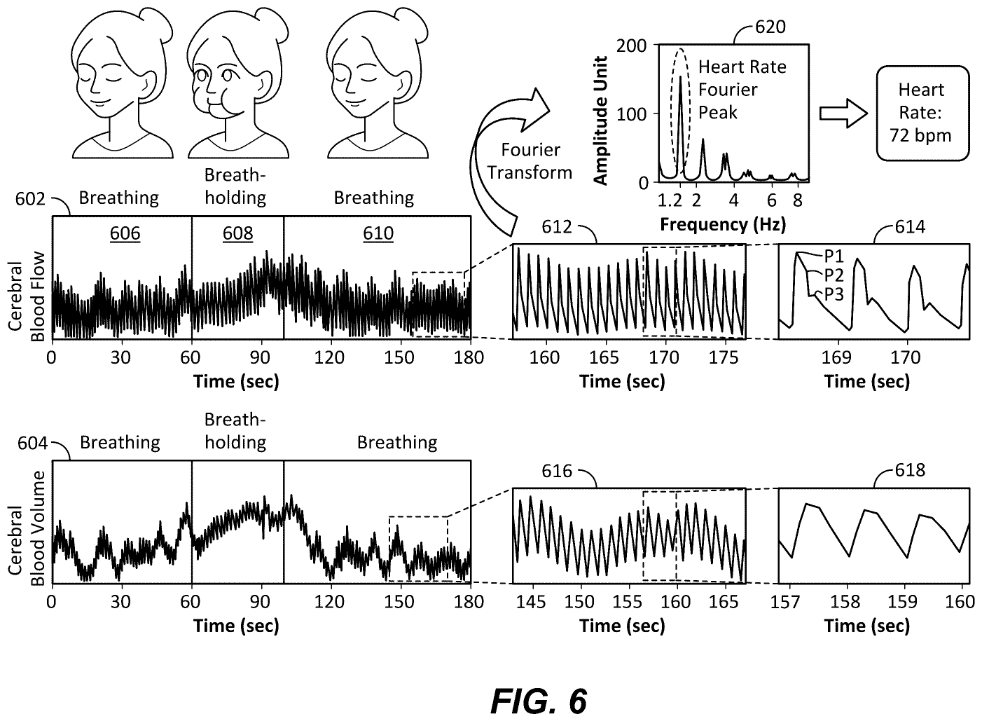

FIG. 1 is a block diagram of an example system for determining stroke likelihood data in accordance with some embodiments. As illustrated, the system includes a stroke prediction engine 102. In some implementations, stroke prediction engine 102 may include a trained machine learning model configured to take, as input, breath-holding data 104, and generate, as an output, stroke likelihood data 104. The trained machine learning model may be a perceptron, a random forest, a deep neural network (DNN), or any other suitable architecture. In instances in which breath-holding data 102 includes raw cerebral blood metric data (e.g., traces of cerebral blood flow, cerebral blood volume, and/or cerebral blood oxygenation as a function of time, as shown in and described below in connection with FIG. 5), the trained machine learning model may be a DNN. Such a DNN may be able to identify features in the cerebral blood metric data not observable or identifiable by a human that are useful for predicting stroke risk. In other embodiments, in instances in which breath-holding data 102 includes extracted features of cerebral blood metric data, (as described in more detail in connection with FIGS. 5 and 6) the trained machine learning model may be a perceptron, a random forest, or other type of architecture configured to take extracted features as input and generate a stroke likelihood. Note that the breath-holding data may be obtained using light emitted into the brain of the patient using one or more light sources and using data representative of absorbed/reflected light using one or more light detectors. Such light emitters and light detectors may be disposed on a headband or headset, as shown in and described in more detail in connection with FIGS. 2A and 2B.

Stroke likelihood data 104 may be a number on a discrete scale (e.g., an integer between 1 and 5), a number on a continuous scale (e.g., a probability value that is a continuous number between 0 and 1), or the like. Stroke likelihood data 104 may represent a likelihood that, given the cerebral blood metrics represented in breath-holding data 104, the patient will have a stroke within a predetermined future time period (e.g., within the next year, within the next five years, within the lifetime of the patient, etc.).

It should be noted that stroke prediction engine 102 may be implemented by one or more computing devices or one or more processors. For example, such a computing device and/or processor may be configured to analyze data from one or more light detectors, generate cerebral blood flow metrics, and/or provide data representative of the cerebral blood flow metrics to a trained machine learning model to generate stroke likelihood data 106. In some implementations, the one or more computing device and/or one or more processors may be disposed on the same headset or headband as the one or more light emitters and one or more light detectors. Additionally, or alternatively, the one or more computing devices and/or one or more processors may be communicatively coupled to the headset or headband, e.g., by a wireless or wired communication channel.

As described above, one or more light emitters and one or more light detectors may be disposed on a head-worn device by a patient. The light may be emitted toward and/or into the head of the patient, thereby probing cerebral blood flow. Reflected light may be captured by the one or more light detectors to assess and/or characterize absorbed and reflected light by structures internal to the patient's brain. In some implementations, a “channel” may be characterized by a light emission package and a corresponding light detector package. In some embodiments, a light emission package may include multiple light emitters, such as a laser and an LED. For example, a laser may emit light in a near infrared or infrared wavelength, and an LED may emit light in a visible, near infrared, or infrared wavelength. Use of multiple wavelengths may allow for cerebral blood oxygenation to be determined. As described below, in some cases, light emitted in an infrared wavelength (e.g., by a laser) may be used to determine cerebral blood volume and/or cerebral blood flow, and light emitted at two different wavelengths (e.g., light emitted in an infrared wavelength and light emitted in a near infrared wavelength) may be used to determined cerebral blood oxygenation. It should be understood that in instances in which two wavelengths of light are used to determined cerebral blood oxygenation, one of the wavelengths may also be used to determine cerebral blood volume and/or cerebral blood flow.

In one particular example, a laser may emit light in a near-infrared wavelength, and the reflected light may be captured by a camera. The reflected light in the near-infrared wavelength range may be used to determine cerebral blood flow and/or cerebral blood volume. Concurrently, an LED may emit light in another near infrared wavelength and the reflected light may be captured by a photodetector. The reflected light from both wavelengths may be used to determined cerebral blood oxygenation. Note that the combination of laser, LED, camera, and photodetector of this example may be considered one “channel,” and a headset may have multiple such channels (e.g., two, four, ten, etc.) disposed at various locations around the headset, each probing a different region of the brain.

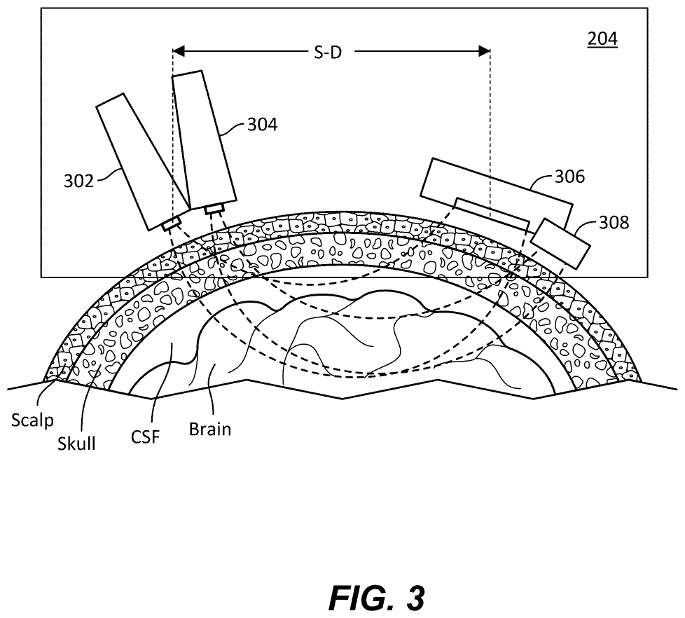

In some embodiments, a light detection package may include multiple light detectors, such as multiple cameras, a camera and a photodetector, or the like. In some implementations, a distance between a light emitter and a corresponding light detector (or between a light emission package comprising two or more light emitters and a corresponding light detection package) may be within a range of about 2.5 cm-4.0 cm. This distance is generally referred to as the “source-detector distance,” and example values may include 2 cm, 2.5 cm, 3 cm, 3.5 cm, 4 cm, etc. In instances in which a headset includes multiple channels (each comprising at least one light emitter or at least one light emission package, and corresponding light detectors or light detection packages), the distance between light emitter and light detector for different channels may be different. In some implementations, positions of light emitters and/or light detectors (or a light emission package and or a light detection package) may be modifiable. For example, a light emission package and/or a light detection package may be affixed to a headband or a headset via screws, Velcro, or other hardware at a position that is modifiable. This may allow for distances between a light source and a light detector to be modified, which may in turn allow the depth of imaging to be modified as different source-detector distances may impact the depth the emitted light can penetrate within the brain, as shown in and described below in connection with FIG. 3. Additionally, modification of positions of light emitters and/or light detectors may allow different brain regions to be probed using one headset.

FIG. 2A illustrates an example headset 202 in accordance with some embodiments. As illustrated, headset 202 includes numerous channels, each comprised of one or more light emitters and one or more light detectors. Each light emitter and each light detector is affixed to a portion of headset 202. As illustrated, headset 202 is configured to encircle at least a portion of the head of a user, e.g., at forehead level. Headset 202 includes channel 204.

Referring to FIG. 2B, as illustrated, channel 204 includes a light emission package 206. Light emission package includes a laser and an LED. As illustrated, channel 204 also includes two light detectors, 208 and 210, which together may be referred to as a “light detection package.” Light detectors 208 and 210 may be cameras, photodiodes, etc.

Note that a headset or headband may include one or more channels (e.g., one, two, four, five, ten, etc.). For example, in some implementations, a headset or headband may include four channels. By way of example, the four channels may be configured to measure cerebral blood metrics on a forehead (or frontal lobe region), a parietal lobe region, or the like. In some embodiments, the channels may be symmetrically disposed with respect to one another. For example, a first channel may be disposed proximate the left parietal lobe, and a second channel may be disposed proximate the right parietal lobe.

FIG. 3 illustrates an example implementation of a channel (e.g., channel 204) in accordance with some embodiments. As illustrated, channel 204 may include a laser 302 and an LED 304. Laser 302 may be configured to emit light in the infrared wavelength. In one example, laser 302 may emit light at about 830 nm. In another example, laser 302 may emit light at about 785 nm. LED 304 may be configured to emit light at a different wavelength than laser 302. In one example, LED 304 may emit light in the near infrared wavelength. Channel 204 also includes two light detectors, camera 306 and light detector 308 (which may be, e.g., a photodiode). As illustrated, camera 306 may obtain light reflected off various brain structures from light emitted by laser 302. Data captured by camera 306 may be used to determine cerebral blood flow and/or cerebral blood volume metrics, as described below in connection with FIGS. 4 and 5. Light detector 308 may obtain light reflected off various brain structures from light emitted by LED 304. Data captured by camera 306 and light detector 308 may be combined to determine cerebral blood oxygenation metrics, as described below in connection with FIG. 4.

In some implementations, cerebral blood metrics may include one or more of cerebral blood flow, cerebral blood volume, and/or cerebral blood oxygenation. In some embodiments, cerebral blood volume may be determined based on the intensity of reflected light as measured by a light detector. Note that cerebral blood volume may be determined based on reflected light of a single wavelength (e.g., emitted from a laser, from an LED, etc.). In some implementations, cerebral blood oxygenation may be determined based on the intensity of reflected light at two wavelengths (e.g., emitted by a laser and an LED), and determining oxygenation based on the differential optical transmission changes for the two different wavelengths. For example, the two wavelengths may be one where oxyhemoglobin absorbs more light and one where deoxyhemoglobin absorbs more light. The two wavelengths may be in the visible, near-infrared, or infrared wavelength range. In some embodiments, known formulas for calculating oxygen saturation (e.g., based on the Beer-Lambert Law, or other similar techniques) may be utilized to determine cerebral blood oxygenation metrics using two wavelengths.

In some embodiments, cerebral blood flow may be determined using collected scattered light at a single wavelength (e.g., light emitted in an infrared wavelength range). Note that wavelength choice may affect penetration depth of the light. For example, light emitted in the infrared wavelength range may penetrate to deeper structures relative to near infrared light. In some implementations, cerebral blood flow may be based on diffuse correlation spectroscopy (DCS), or on speckle visibility spectroscopy (SVS), which is also referred to as speckle contrast optical spectroscopy (SCOS). In general, a “speckle” refers to the pattern of bright and dark spots in an image resulting from scattering of illuminated laser light (e.g., scattered by the scalp, skull, and/or brain) resulting from constructive and destructive interference of the light. The speckle pattern dynamics change with blood flow dynamics. The time that it takes one speckle pattern to change to a different speckle pattern is referred to as the decorrelation time, which is correlated with cerebral blood flow rate. Both DCS and SCOS are techniques for measuring how fast the speckle pattern changes, and hence, estimating cerebral blood flow rate. DCS may determine changes in speckle decorrelation time based on a single pixel of the speckle image, i.e., requiring a fast photodetector to detect the light. In contrast, SCOS may determine speckle decorrelation time using a relatively slow camera with a large number of pixels. In particular, the camera exposure time, typically in the range of 0.3 milliseconds to 15 milliseconds, may be set to be substantially longer than the decorrelation time of the speckle field (typically a few tens of microseconds), which may result in multiple different speckle patterns summed up into a single camera frame. As the speckle field fluctuates, the speckle pattern recorded by the camera is smeared and washed out within the exposure time. Accordingly, the cerebral blood flow may be quantified from the degree of blurring of the captured frame, which is generally referred to herein as the speckle contrast. Note that use of SCOS to measure cerebral blood flow may have the advantage that a relatively inexpensive camera may be used as a light detector, because a high frame rate is not needed, unlike detectors used for DCS. Moreover, the camera may be directly mounted on a headset or headband, which may eliminate optical loss associated with a light guide running from the head to the camera, which may introduce its own noise and motion artifacts. In some examples, a camera used for SCOS may have an integration time of within a range of about 0.3 milliseconds-15 milliseconds. The camera may use a frame rate of within a range of about 30 frames per second-150 frames per second. In one example, the camera may have a frame rate of about 80 frames per second.

Speckle contrast may be determined at each camera frame as:

$$K = \frac{{σ}_{1}}{\overline{I}} = \frac{\sqrt{< {I}_{j}^{2} > - < {I}_{j} {>}^{2}}}{< {I}_{j} >}$$

In the equation given above, Iⱼ represents the instantaneous intensity recorded at the camera at pixel J, or represents the standard deviation of I, and Ī represents the mean of I. The speckle decorrelation time is determined as:

$$r C B F ∝ \frac{1}{τ} = \frac{1}{β {K}^{2}}$$

The speckle decorrelation time is directly correlated with the cerebral blood flow rate. The relative cerebral blood flow, measured in units of blood flow index (BFI), is inversely correlated with t and therefore inversely correlated with βK². The correction factor β is a constant depending on system setup, e.g. speckle size, pixel size, polarization of the laser light.

FIG. 4 illustrates use of DCS and SCOS for determining cerebral blood flow in accordance with some embodiments. As illustrated, DCS samples a single fluctuating speckle at a relatively high rate, and calculates the cerebral blood flow from the time trace of the intensity at a single point. In particular, graph 402 illustrates the fluctuating intensity as a function of time of a speckle at a single point, and graph 404 illustrates the cerebral blood flow rate determined based on an auto correlation of the fluctuating intensity depicted in graph 402. SCOS captures an integrated speckle pattern over time, where the speckle fluctuates more quickly over time at higher cerebral blood flow rates, leading to washout in the captured image. By measuring the extent of the washout or blurring in the captured image (e.g., the speckle contrast), cerebral blood flow can be determined. Images 406 illustrate the speckle patterns that are integrated in a single image to determine speckle contrast, which is used to determine cerebral blood flow as a function of time, as illustrated in graph 408.

FIG. 5 illustrates example graphs of cerebral blood volume, cerebral blood flow, and cerebral blood oxygenation as a function of time in accordance with some embodiments. As described above, these cerebral blood metrics may be obtained over a period of time, where, during a subset of the period of time, the patient is holding their breath. Breath holding time period 502 is illustrated in FIG. 5. Note that, cerebral blood metrics may be obtained over a baseline time period, generally referring to the time period during which cerebral blood metrics are obtained prior to initiation of breath holding. Baseline time period 504 is illustrated in FIG. 5. Cerebral blood metrics may also be obtained during a recovery time period 506, which generally begins at a time from when breath holding ends (e.g., the end of breath holding time period 502).

Note that each time period may be any suitable duration of time. Example duration of breath holding may be 15 seconds, 30 seconds, 45 seconds, 60 seconds, etc. In some embodiments, the duration of breath holding may be however a long a patient can hold their breath relatively comfortably (e.g., the breath holding time period may vary for different people). In some implementations, the baseline time period and/or the recovery time period may be at least as long as the breath holding time period. In some embodiments, the recovery time period may be longer than the breath holding time period. In some embodiments, the recovery time period may be a time duration long enough that cerebral blood flow metrics return to within a predetermined range of the corresponding values during the baseline time period. In some such embodiments, the recovery time period may be dynamically adjusted. For example, the recovery time period may be stopped responsive to determined that cerebral blood metrics have returned to baseline values.

FIG. 6 illustrates examples of experimental cerebral blood metric data collected during a breath holding task in accordance with some embodiments. As illustrated by graph 602, cerebral blood flow is determined during a time period that includes the patient holding their breath. Similarly, as illustrated by graph 604, cerebral blood volume is determined over the same time period. Note that cerebral blood flow and cerebral blood volume may be determined using light emitted at a single wavelength (e.g., in the infrared wavelength region) and the captured reflected light data at the single wavelength. As described above, cerebral blood flow may be determined using based on speckle decorrelation time (e.g., using DCS or SCOS, as described above), and cerebral blood volume may be determined based on the intensity of the reflected light. In the example data shown in FIG. 6, cerebral blood flow is determined using SCOS. Note that graphs 602 and 604 indicate a baseline time period 606 prior to initiation of breath holding, a breath holding time period 608 during which time the patient is holding their breath, and a recovery time period 610 after the patient resumes normal breathing.

As illustrated in graphs 602 and 604, both cerebral blood flow and cerebral blood volume increase during breath holding time period 608. This is attributed to the brain's increased demand for blood to transport oxygen and carbon dioxide until breath holding stops. During the breath holding time period 608, the brain enters a heightened state of alert which triggers a sequence of protective mechanisms to ensure stable regulation of carbon dioxide and oxygen, which is achieved through accelerated circulation of blood leading to increased blood flow together with an increase in blood volume in the brain via dilation of blood vessels.

Additionally, note that both cerebral blood flow rates and cerebral blood volume remain elevated at the beginning of recovery time period 610 prior to recovery to the baseline level of each metric.

Plots 612 and 614 illustrate subsets of cerebral blood flow graph 602 over short time scales. Similarly, plots 616 and 618 illustrate subsets of cerebral blood volume graph 604 over short time scales. Note that the pulsations evident in each of plots 612, 614, 616, and 618 are not noise but represent blood pulsations. As illustrated in frequency domain graph 620, the pulsations may be used to determine a heart rate of the patient (e.g., based on the periodicity of the pulsations). For example, heart rate may be determined by taking a Fourier transform of time domain data. Additionally, note that cerebral blood flow and cerebral blood volume may capture different details in blood flow. For example, the dicrotic notch and peak pressure are discernible in plot 614 of the cerebral blood flow.

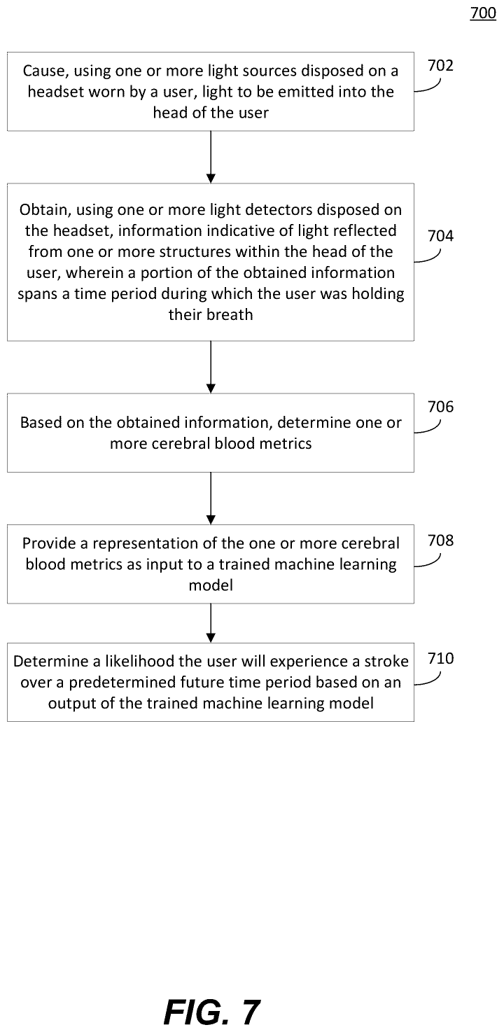

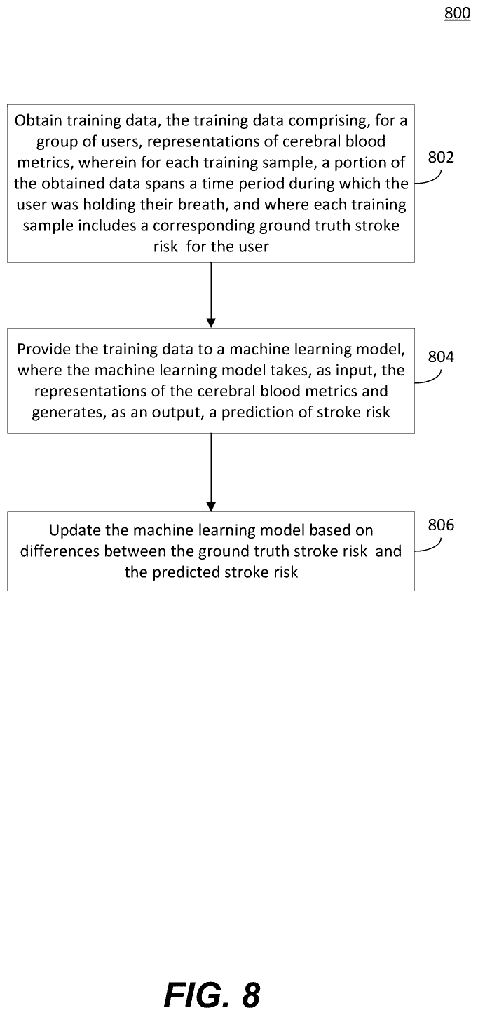

In some implementations, raw data traces of cerebral blood metrics (e.g., cerebral blood volume, cerebral blood flow, and/or cerebral blood oxygenation as a function of time) may be provided to a trained machine learning model configured to output a stroke likelihood prediction. Such a machine learning model that accepts raw data traces may be a DNN or other suitable architecture. Alternatively, in some embodiments, one or more features associated with the cerebral blood metrics may be extracted. The extracted features may then be provided to a trained machine learning model, which in turn may generate a predicted stroke likelihood. The extracted features may be considered “a representation of the one or more cerebral blood flow metrics.” In instances in which one or more extracted features are provided to a machine learning model, the model may be a perceptron, a random forest, or any other suitable architecture. Note that techniques for generating predicted stroke likelihoods using a machine learning model are shown in and described below in connection with FIG. 7, and techniques for training such a model are shown in and described below in connection with FIG. 8.

In some implementations, extracted features may include information a duration of time a patient was able to hold their breath (generally referred to herein as T_{BH}).

In some implementations, extracted features may include percentage change in a cerebral blood metric at a maximum or minimum after initiation of breath holding compared to a baseline value. For example, referring to FIG. 5, the percentage change in cerebral blood volume (CBV change), the percentage change in cerebral blood flow (CBF change), and the percentage change in cerebral blood oxygenation (CBO change) relative to the baseline value) are indicated. Note that to determine a percentage change, each cerebral blood metric may be normalized based on the baseline value.

In some implementations, the extracted features may include a rate of change (e.g., a slope) in the change in a cerebral blood metric during breath holding. For example, rate of change of cerebral blood flow may be determined by dividing a percentage change of cerebral blood flow (e.g., as indicated in FIG. 5) by the duration of time the patient holds their breath, to derive a feature with units of percent change per second. Similarly, an extracted feature may include a rate of recovery (e.g., a slope) in the change in a cerebral blood metric after resuming normal breathing. Note that, in some implementations, rates of change either during the breath holding time period, or a rate of change associated with recovery, may be determined by fitting a function (e.g., an exponential function) to a portion of the cerebral blood metric, and estimating rate of change metrics based on a growth or decay constants from the fitted function.

In some implementations, the extracted features may include a ratio of a percentage change of one cerebral blood metric to a percentage change of another cerebral blood metric. In one example, an extracted feature may be a ratio of the percentage change of cerebral blood flow (e.g., CBF change in FIG. 5) to the percentage change of cerebral blood volume (e.g., CBV change in FIG. 5). Other examples include a ratio of the percentage change of cerebral blood flow to cerebral blood oxygenation, and/or a percentage change of cerebral blood volume to cerebral blood oxygenation. In some implementations, the extracted features may include a ratio of rate of change of one cerebral blood metric to a rate of change of another cerebral blood metric. Examples include a rate of change of cerebral blood flow to a rate of change of cerebral blood volume, a rate of change of cerebral blood flow to a rate of change of cerebral blood oxygenation, or a rate of change of a cerebral blood volume to a rate of change of cerebral blood oxygenation. Note that ratios of rates of change may be determined based on rate of change either during the breath holding time period, or during a recovery time period after normal breathing resumes.

In some implementations, extracted features may include fine grained features from within a cardiac cycle as observed within a cerebral blood flow trace. For example, referring to FIG. 6, graph 614 illustrates three peaks, labeled P1, P2, and P3, within the cerebral blood flow trace in graph 614. Note that all of P1, P2, and P3 are from within a single cardiac cycle, where the cerebral blood flow trace includes multiple (e.g., hundreds) of cardiac cycles, each with their own peaks. In general, P1 corresponds to the rapid ejection of blood during systole, the second peak P2 corresponds to reflected waves from the vascular tree, and P3 corresponds to the dicrotic notch at the beginning of diastole. In some embodiments, ratios of the amplitude of any of these peaks (P1 to P2, P2 to P3, and/or P1 to P3) may be used to form an extracted feature. The ratio may be taken from a cardiac cycle extracted from the baseline time period, during the breath holding time period, or during the recovery time period. In some implementations, a ratio of a peak ratio from the breath holding time to a peak ratio from a baseline or recovery time period may be considered an extracted feature. By way of example, the ratio of the P2 peak to the P1 peak during breath holding may be determined and represented as the breath holding peak ratio. Continuing with this example, the ratio of the P2 peak to the P1 peak during the baseline time period may be determined and represented as the baseline peak ratio. An extracted feature may then be the breath holding peak ratio to the baseline peak ratio.

Note that extracted features may be extracted and/or determined autonomously (e.g., without user input) upon collection of the one or more cerebral blood flow metrics. The extracted features may then be provided to a trained machine learning model configured to generate the prediction of stroke likelihood for the patient. Note that any suitable number or combination of extracted features may be utilized.

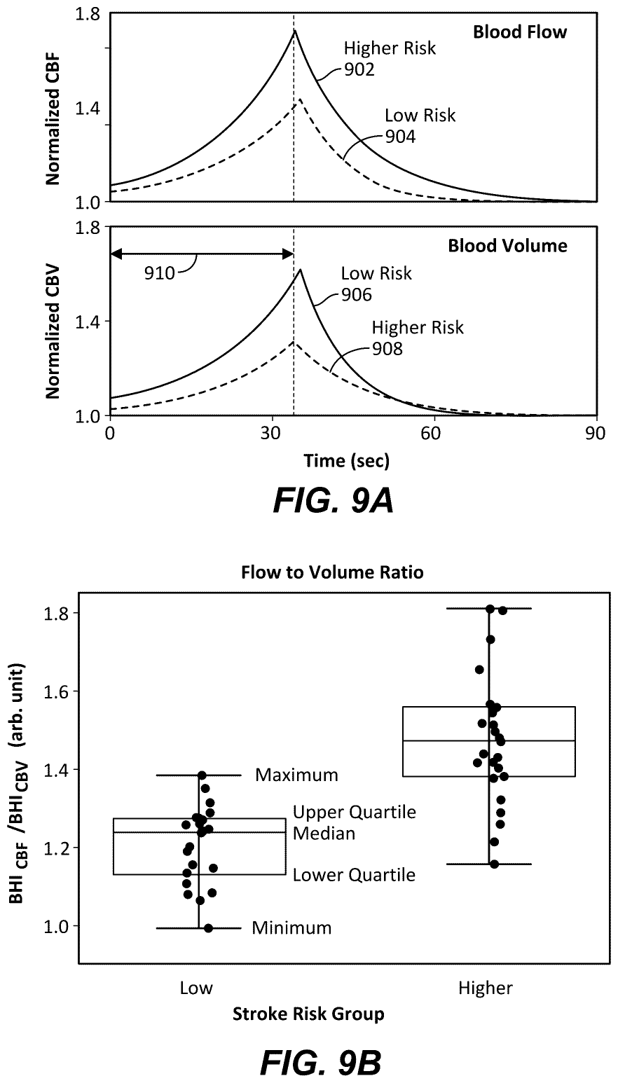

Alternatively, in some embodiments, rather than utilizing a trained machine learning model, stroke risk may be predicted based on extracted features, e.g., by comparing an extracted feature to one or more predetermined thresholds. By way of example, stroke risk may be classified as “high,” responsive to determining that a value of a particular extracted feature (e.g., the percentage change in cerebral blood flow) exceeds a predetermined threshold. As another example, stroke risk may be classified as “high” responsive to determining that a value of a particular extracted feature (e.g., the percentage change in cerebral blood volume) is below a predetermined threshold. In one example, stroke likelihood may be determined based on a ratio of a percentage change in cerebral blood flow (between a peak cerebral blood flow value after the user begins holding their breath and a baseline cerebral blood flow to a percentage change in cerebral blood volume (between a peak cerebral blood volume after the user begins holding their breath and a baseline cerebral blood volume), as shown in and described below in connection with FIGS. 9A and 9B. In particular, the ratio may be determined, and in some cases, compared to a predetermined threshold to generate a stroke likelihood. In some cases, multiple extracted features may be considered and may be compared to predetermined thresholds, and a stroke risk may be determined based on an aggregation of the multiple extracted features. For example, a stroke risk may be categorized as “high” responsive to more than 50%, 60%, 70%, etc. of extracted features meeting threshold criteria for high stroke risk. Note that, in such instances, a computing device may store thresholds for categorizing stroke risk based on any suitable extracted features, and may perform such comparisons to generate a stroke risk. Such a computing device may be disposed on a headset, or may be communicatively coupled to a processor or other computing device disposed on the headset.

In one example, extracted features may be based on a breath holding index (BHI), generally defined herein as the maximal change from baseline during breath-holding divided by the duration of breath-holding T_{BH} for a given cerebral blood metric. For example, BHI for cerebral blood flow may be defined by:

$${BHI}_{C B F} = 100 * \frac{{CBFI}_{max} - {CBFI}_{0}}{{CBFI}_{0} * {T}_{B H}}$$

Similarly, the BHI for cerebral blood volume may be defined by:

$${BHI}_{C B V} = 100 * \frac{{CBVI}_{max} - {CBVI}_{0}}{{CBVI}_{0} * {T}_{B H}}$$

In some examples, the flow to volume ratio may be an extracted feature used to predict stroke risk. The flow to volume ratio may be determined based on the ratio of the BHI for cerebral blood flow to the BHI for cerebral blood volume:

$$Flow to volume ratio : \frac{{BHI}_{C B F}}{{BHI}_{C B V}}$$

In some cases, BHI_{CBF} may have a value within a range of about 0% to 5%, and BHI_{CBV} may have a value within a range of about 0% to 3%. The flow to volume ratio may have a value within a range of about 0.9 to 2. A threshold for categorizing a patient as having relatively high stroke risk may be based on the flow to volume ratio (e.g., the patient may be categorized as high risk if the flow to volume ratio is greater than 1.2, greater than 1.3, greater than 1.4, etc.) FIG. 9B presents experimental data depicting the flow to volume ratio using the BHI for patients deemed to be high risk or low risk based on stroke questionnaire data.

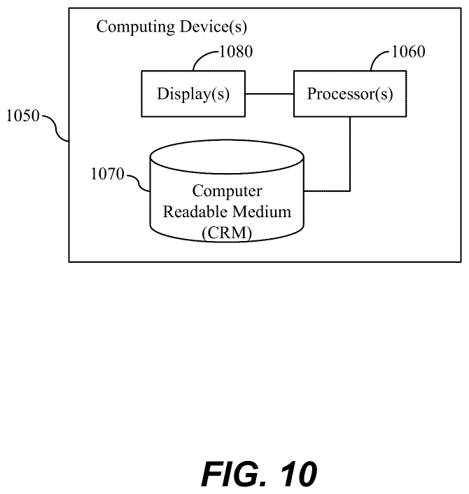

FIG. 7 is a flowchart of an example process 700 for determining a stroke likelihood using cerebral blood metrics in accordance with some embodiments. Blocks of process 700 may be executed by one or more processors of one or more computing devices. An example of such a computing device is shown in and described below in connection with FIG. 10. Note that, in some embodiments, at least one of the one or more computing devices may be disposed on a headband or headset on which the one or more light sources and light detectors are disposed. Accordingly, in some such embodiments, cerebral blood metrics, extracted features associated with the cerebral blood metrics, and/or the stroke likelihood may be determined by a computing device itself on the headband or headset. Alternatively, in some embodiments, data obtained by the one or more light detectors, and/or data representative of the cerebral blood metrics may be transmitted from a computing device disposed on the headband or headset to a second computing device remote from or separate from the headband or headset, where the second computing device generates the stroke likelihood. In some implementations, blocks of process 700 may be executed in an order other than what is shown in FIG. 7. In some embodiments, one or more blocks of process 700 may be omitted, and/or two or more blocks may be executed substantially in parallel.

Process 700 can begin at 702 by causing, using one or more light sources disposed on a headset worn by a user, light to be emitted into the head of the user. An example of such a headset is shown in and described above in connection with FIGS. 2A and 2B. The one or more light sources may include one or more lasers, one or more LEDs, etc. As described above, in some implementations, two light sources of different types (e.g., a laser and an LED), each of which may emit light in a different wavelength region (e.g., infrared and near infrared) may be packaged together as a light emission package. In some implementations, multiple light emission packages may be disposed on a headset or headband, each configured to emit light into different regions of the user's head or brain. Note that light may be emitted continuously, or may be pulsed.

At 704, process 700 may obtain, using one or more light detectors disposed on the headset, information indicative of light reflected from one or more structures within the head or brain of the user, wherein a portion of the obtained information spans a time period during which the user was holding their breath. An example of one or more light detectors disposed on a headset is shown in and described above in connection with FIGS. 2A and 2B. As described above, the one or more light detectors may include a camera, a photodetector, etc. In instances in which a light emission package includes two light sources each emitting light in a different wavelength range, a corresponding light detection package may include two light detectors, each configured to receive reflected light corresponding to emissions from the corresponding light emitter. In one example, light emitted by a laser may be reflected from various head and brain structures and may be captured by a camera (e.g., to determine speckle contrast, as described above), and light emitted by an LED may be reflected and captured by a photodetector. Note that, as shown in and described above in connection with FIGS. 2A and 2B, because a headset may include multiple (e.g., two, four, eight, ten, etc.) light emission packages and corresponding light detection packages, the obtained information may correspond to different brain regions (e.g., a left frontal lobe region, a right frontal lobe region, a left parietal lobe region, a right parietal lobe region, etc.).

Note that, as described above in connection with FIGS. 5 and 6, the obtained light reflection data spans a baseline time period, a breath holding time period, and a recovery time period. In some implementations, process 700 may cue the user to begin holding their breath at a particular time. For example, the cue may be an audible cue (e.g., a spoken instruction, an audible beep, etc.), or may be a haptic cue (e.g., a vibration delivered using the headset).

At 706, process 700 can, based on the obtained information, determine one or more cerebral blood metrics. As described above, the cerebral blood metrics may include cerebral blood volume, cerebral blood flow, and/or cerebral blood oxygenation. Process 700 may additionally determine a duration of time the user held their breath. The duration of time may be determined by, e.g., thresholding any of the cerebral blood metrics to determine a time point at which the cerebral blood metric began deviating from baseline to the time point at which the cerebral blood metric reached a minimum or maximum value. Note that, as described above, cerebral blood volume may be determined based on intensity of the reflected light signal at a single wavelength. Cerebral blood flow may be determined using DCS and/or SCOS (as shown in and described above in connection with FIG. 4). Cerebral blood oxygenation may be determined based on the ratio of reflected light at two different wavelengths (e.g., in instances in which light is emitted by at least two light sources at two different wavelengths, such as an infrared wavelength and a near infrared wavelength).

At 708, process 700 can provide a representation of the one or more cerebral blood metrics as input to a trained machine learning model. Note that the representation of the one or more cerebral blood metrics may include the raw data of the cerebral blood metrics as a function of time, or, alternatively, may include one or more extracted features extracted from the one or more cerebral blood metrics. Examples of extracted features are described above.

At 710, process 700 can determine a likelihood the user will experience a stroke over a predetermined future time period based on an output of the trained machine learning model. For example, the likelihood may correspond to a time period of the next year, the next five years, the next ten years, the remainder of their lifetime, etc. The likelihood may be provided as a discrete classification (e.g., low risk, medium risk, high risk), a continuous value (e.g., a continuous probability value), or in any other suitable format.

In some implementations, the stroke likelihood may be presented in any suitable manner. For example, the stroke likelihood may be audibly presented (e.g., by speakers associated with the headset or headband, or by speakers of an associated or paired device). The stroke likelihood may be visibly presented, e.g., on a display of an associated or paired device. In some implementations, the stroke likelihood may be automatically (e.g., without user input) stored in an electronic medical record associated with a patient, e.g., such that a physician can review the stroke likelihood. In cases in which the stroke likelihood is stored, e.g., as medical data, the stroke likelihood may be stored in conjunction with timestamp information indicating a date and/or time the stroke likelihood prediction was made. This may allow a physician or other healthcare provider to monitor changes in the stroke likelihood for a given patient over time. This may allow the healthcare provider to determine whether various interventions are modifying stroke likelihood (e.g., lowering the risk of stroke) over time. In some implementations, an updated stroke likelihood may be determined after a first stroke likelihood is determined (e.g., two months later, six months later, a year later, two years later, etc.) A difference between the first stroke likelihood and the updated stroke likelihood may be determined, e.g., to determine if the patient's likelihood of experiencing stroke remains the same over time, is increasing over time, or is decreasing over time. In some embodiments, a recommendation may be generated based on the change in stroke likelihood over time. For example, a recommendation to continue or implement particular lifestyle modifications may be made, a recommendation to initiate a particular medical treatment may be made, etc. In some embodiments, an indication of the change in stroke likelihood over time may be transmitted to a user device associated with the patient, with a healthcare provider, etc.

A machine learning model may be trained using a training set that includes representations of cerebral blood metrics (which may include cerebral blood flow, cerebral blood volume, and/or cerebral blood oxygenation as a function of time, or extracted features as discussed above), and corresponding ground truth data. The training set may include data associated with multiple patients, who may be of varying demographics (e.g., different lifestyles, different ages, different sexes, etc.). In some embodiments, ground truth data may be questionnaire data, where the questionnaire predicts a stroke likelihood based on demographic data, lifestyle data, physiological metrics (e.g., blood pressure, resting heart rate, etc.), and the like. Alternatively, in some embodiments, ground truth data may be actual stroke occurrence data, e.g., obtained from longitudinal data that collects representations of cerebral blood metrics from a set of patients who are followed over time. Continuing with this example, the ground truth data may indicate that the patient did not have a stroke if no stroke was recorded during the duration of time the longitudinal study occurred, or, conversely, may indicate that the patient did have a stroke if such stroke occurred. Regardless of how ground truth data is obtained, a machine learning model may be trained by providing representations of cerebral blood flow metrics as input, obtaining a stroke risk prediction based on the input, and updating weights of the model based on a difference between the predicted stroke risk and the ground truth stroke risk. This procedure may be implemented to train a DNN that operates on traces of cerebral blood metrics as a function of time, and/or a perceptron, random forest, or other type of network that operates on extracted features associated with cerebral blood metrics or associated with aspects of the breath holding task (such as duration of breath holding).

FIG. 8s a flowchart of an example process 800 for training a machine learning model in accordance with some embodiments. Blocks of process 800 may be executed by one or more computing devices, such as a server device, a desktop computer, a laptop computer, etc. Note that the one or more computing devices may be different than the one or more computing devices that execute blocks of process 700. In some implementations, blocks of process 800 may be executed in an order other than what is shown in FIG. 8. In some embodiments, two or more blocks of process 800 may be executed substantially in parallel. In some embodiments, one or more blocks of process 800 may be omitted.

Process 800 can begin at 802 by obtaining training data, the training data comprising, for a group of users, representations of cerebral blood metrics, wherein for each training sample, a portion of the obtained data spans a time period during which the user was holding their breath, and where each training sample includes a corresponding ground truth stroke risk for the user. As described above, the representations of cerebral blood metrics may include one or more cerebral blood metrics (e.g., cerebral blood flow, cerebral blood volume, and/or cerebral blood oxygenation) as a function of time, and/or extracted features associated with the cerebral blood metrics and/or the breath holding task. Note that cerebral blood metrics for the training data may have been collected using one or more light sources and/or one or more light detectors disposed on a headset or headband similar to the one shown in and described above in connection with FIGS. 2A and 2B. The cerebral blood metrics may be determined based on collected light reflectance data as described above. Ground truth stroke risk data may be questionnaire based, or may be actual stroke occurrence data based on a longitudinal following of the users over time.

At 804, process 800 can provide the training data to a machine learning model, where the machine learning model takes, as input, the representations of the cerebral blood metrics and generates, as an output, a prediction of stroke risk.

At 806, process 800 can update the machine learning model based on differences between the ground truth stroke risk and the predicted stroke risk. For example, weights of the model may be updated based on a loss function that considers the difference between the ground truth stroke risk and the predicted stroke risk. Note that model updating may be performed for each training sample, or for a batch of training samples.

Note that data (e.g., cerebral blood metric data) included in the training set may be obtained using a headset or head-worn device, and the trained machine learning model resulting from such a training set may be used to generate stroke risk predictions based on cerebral blood metrics obtained using the same headset or head-worn device, or a different but similar headset or head-worn device. For example, a similar device may be one in which light emitted by one or more light emitters is at about the same wavelength, where source-detector distance is about the same, or the like.

FIGS. 9A and 9B illustrates experimental data in accordance with some embodiments. To obtain the experimental data depicted in FIGS. 9A and 9B, cerebral blood flow (labeled CBF) and cerebral blood volume (labeled CBV) for obtained for 50 patients. The patients were divided into a “low risk” group and a “higher risk” group, determined based on stroke questionnaire data. CBF and CBV were determined using the light emittance and light reflectance techniques described above, in particular, using a laser emitting infrared light, and a camera configured to obtain reflected light. Cerebral blood flow was determined using SCOS, and cerebral blood volume was determined based on intensity of captured reflected light.

As illustrated in FIG. 9A, curve 902 depicts the cerebral blood flow for patients deemed high risk, and curve 904 depicts the cerebral blood flow for patients deemed low risk. The breath holding time period 910 is marked. Note that curves 902 and 904 are generated by normalizing and averaging cerebral blood flow across all of the patients in a given risk group. Note that the maximal cerebral blood flow is higher for the patients deemed high risk (represented in the higher peak in cerebral blood flow for the higher risk group) compared to the low risk group. This is presumed to be due to the less flexible blood vessels of the high risk group, which causes an impedance in blood vessel dilation during breath holding, which triggers accelerated blood flow.

Conversely, as shown by curves 906 and 908, the cerebral blood volume for the low risk group peaks at a higher level than for the high risk group. The difference in peaks in cerebral blood flow and cerebral blood volume (i.e., the percentage change in cerebral blood flow from baseline and the percentage change in cerebral blood volume from baseline) were found to be significant across the low risk and high risk groups, and accordingly, may be strong predictors of stroke risk. Additionally, note that because the peak change is in opposite directions for cerebral blood flow versus cerebral blood volume (in other words, the low risk group had a higher cerebral blood volume and a lower cerebral blood flow, and vice versa for the high risk group), the ratio of the percentage change of cerebral blood flow to percentage change of cerebral blood volume was found to be a highly significant predictor of stroke risk.

FIG. 9B illustrates box plots of the ratio of the percentage change of cerebral blood flow to the percentage change of cerebral blood volume for low and high risk groups in box plot form. For the data shown, percentage change is measured from the peak after initiation of breath holding to a baseline value using the BHI, as described above, for both cerebral blood flow and cerebral blood volume. The ratio is determined as the BHI of cerebral blood flow to the BHI of cerebral blood volume. The data illustrated in FIG. 9B show a statistically significant difference between patients assessed to be at low risk of stroke and those assessed to be at high risk of stroke, validating that the ratio of percentage change of cerebral blood flow to the percentage change of cerebral blood volume is a robust predictor of stroke risk.

### Computational Systems

The techniques described above may be implemented using one or more computing devices. For example, a machine learning model may be trained and/or utilized at inference time using a computational device such as a server device, a laptop computer, a desktop computer, or the like. FIG. 10 illustrates an example computing device that may be used, e.g., to implement blocks of process 700 and/or 800 of FIGS. 7 and/or 8, respectively. Note that such a computing device may be part of a headset comprising one or more light sources and/or one or more light detectors (e.g., the computing device may be disposed on a portion of the headset or headband), or may be communicatively coupled to the headset (e.g., via a wireless communication channel, such as BLUETOOTH).

In FIG. 10, the computing device(s) 1050 includes one or more processors 1060 (e.g., microprocessors), a non-transitory computer readable medium (CRM) 1070 in communication with the processor(s) 1060, and one or more displays 1080 also in communication with processor(s) 1060.

Processor(s) 1060 is in electronic communication with CRM 1070 (e.g., memory). Processor(s) 1060 is also in electronic communication with display(s) 1080, e.g., to display image data, text, etc. on display 1080.

Processor(s) 1060 may retrieve and execute instructions stored on the CRM 1070 to perform one or more functions described above. For example, processor(s) 1060 may execute instructions to perform one or more operations to analyze collected data (e.g., light reflection/absorption data), provide collected data to a trained model, train a machine learning model to generate a stroke risk prediction, etc.

The CRM (e.g., memory) 1070 can store instructions for performing one or more functions of the described above. These instructions may be executable by processor(s) 1070. CRM 1070 can also store raw images, e.g., speckle images, or the like.

### EXAMPLE EMBODIMENTS

Embodiment 1: A method of determining stroke risk, the method comprising: causing, using one or more light sources disposed on a headset worn by a user, light to be emitted into the head of the user; obtaining, using one or more light detectors disposed on the headset, information indicative of light reflected from one more structures within the head of the user, wherein a portion of the obtained information spans a time period during which the user was holding their breath; based on the obtained information, determining one or more cerebral blood metrics; providing a representation of the one or more cerebral blood metrics as input to a trained machine learning model; and determining a likelihood the user will experience a stroke over a predetermined future time period based on an output of the trained machine learning model.

Embodiment 2: The method of embodiment 1, further comprising: determining an updated likelihood the user will experience a stroke based on updated cerebral blood metrics; and determining a change between the likelihood and the updated likelihood.

Embodiment 3: The method of embodiment 2, further comprising providing at least one recommendation based on the change between the likelihood and the updated likelihood.

Embodiment 4: The method of any one of embodiments 1-3, wherein the one or more light sources comprise at least two light sources, each configured to emit light in a different wavelength.

Embodiment 5: The method of claim 4, wherein the at least two light sources comprise a laser and a light emitting diode packaged together.

Embodiment 6: The method of any one of embodiments 4 or 5, wherein the at least two light sources comprise a laser configured to emit light in an infrared wavelength range.

Embodiment 7: The method of any one of embodiments 4-6, wherein the at least two light sources comprise a light emitting diode configured to emit light in a near-infrared wavelength range.

Embodiment 8: The method of any one of embodiments 1-7, wherein the one or more light detectors comprise at least two detectors of different types.

Embodiment 9: The method of embodiment 8, wherein the at least two detectors of different types comprise at least one camera.

Embodiment 10: The method of embodiment 9, wherein the at least one camera is configured to capture images comprising a speckle pattern.

Embodiment 11: The method of any one of embodiments 1-10, wherein the one or more cerebral blood metrics comprise at least one of: cerebral blood flow, cerebral blood oxygenation, or cerebral blood volume.

Embodiment 12: The method of embodiment 11, wherein the cerebral blood flow is determined based on a decorrelation time associated with a series of speckle patterns obtained from a series of images captured by at least one camera included in the one or more light detectors.

Embodiment 13: The method of any one of embodiments 11 or 12, wherein the representation of the obtained information comprises a ratio of change in cerebral blood flow during the time period the user was holding their breath to a baseline period to a change in cerebral blood volume during the time period the user was holding their breath to a baseline period.

Embodiment 14: The method of any one of embodiments 1-13, wherein providing the representation of the one or more cerebral blood metrics as input comprises providing raw traces of the one or more cerebral blood metrics as a function of time to the trained machine learning model, and wherein the trained machine learning model is a deep neural network (DNN).

Embodiment 15: The method of any one of embodiments 1-14, wherein providing the representation of the one or more cerebral blood metrics as input comprises providing features of the one or more cerebral blood metrics as an input to trained machine learning model.

Embodiment 16: A system for determining stroke risk, the system comprising: a headband configured to encircle a head of a wearer of a headset; a plurality of light sources attached to the headband; a plurality of light detectors attached to the headband; and one or more processors. The one or more processors may be configured to: cause, using the one or more light sources, light to be emitted into the head of the wearer; obtain, using the one or more light detectors, information indicative of light reflected from one more structures within the head of the wearer, wherein a portion of the obtained information spans a time period during which the wearer was holding their breath; based on the obtained information, determine one or more cerebral blood metrics; determine a likelihood the wearer will experience a stroke over a predetermined future time period based on the one or more cerebral blood metrics.

Embodiment 17: The system of embodiment 16, wherein the plurality of light sources comprise a plurality of light emission packages, each light emission package comprising at least two light sources configured to emit light in different wavelengths.

Embodiment 18: The system of embodiment 17, wherein the at least two light sources comprise a laser configured to emit light in an infrared wavelength range, and a light emitting diode (LED) configured to emit light in a near infrared wavelength range.

Embodiment 19: The system of any one of embodiments 16-18, wherein the plurality of light detectors comprise a plurality of light detection packages, each light detection package comprising at least two light detectors.

Embodiment 20: The system of embodiment 19, wherein a light detector of the at least two light detectors of a light detection package comprises a camera.

Embodiment 21: The system of embodiment 20, wherein the obtained information comprises a speckle pattern obtained using images captured by the camera.

Embodiment 22: The system of embodiment 21, wherein the one or more cerebral blood metrics comprise a cerebral blood flow determined based on the speckle pattern.

Embodiment 23: The system of any one of embodiments 16-22, wherein a distance between a light source of the one or more light sources and a light detector of the one or more light detectors is adjustable by changing a position of the light source and/or a light detector on the headband.

Embodiment 24: A method of determining stroke risk, the method comprising: causing, using one or more light sources disposed on a headset worn by a user, light to be emitted into the head of the user; obtaining, using one or more light detectors disposed on the headset, information indicative of light reflected from one more structures within the head of the user, wherein a portion of the obtained information spans a time period during which the user was holding their breath; based on the obtained information, determining a cerebral blood flow as a function of time and a cerebral blood volume as a function of time, wherein the cerebral blood flow and the cerebral blood volume include the time period during which the user was holding their breath and a baseline time period before the user was holding their breath; and determining a likelihood the user will experience a stroke over a predetermined future time period based on the cerebral blood flow and the cerebral blood volume.

Embodiment 25: The method of embodiment 24, wherein the likelihood the user will experience the stroke is based on a ratio of a percentage change of cerebral blood flow from a peak after the user began holding their breath to a baseline cerebral blood flow during the baseline time period to a percentage change of cerebral blood volume from a peak after the user began holding their breath to a baseline cerebral blood volume during the baseline time period.

Embodiment 26: A method of training a machine learning model to predict stroke risk, the method comprising: obtaining training data, the training data comprising, for a group of users, representations of cerebral blood metrics, wherein for each training sample, a portion of the obtained data spans a time period during which the user was holding their breath, and wherein each training sample includes a corresponding ground truth stroke risk for the user; providing the training data to a machine learning model, wherein the machine learning model takes, as input, the representations of the cerebral blood metrics and generates, as an output, a prediction of stroke risk; and updating the machine learning model based on differences between the ground truth stroke risk and the predicted stroke risk to generate a trained machine learning model configured to predict stroke risk.

Embodiment 27: The method of embodiment 26, wherein the representations of cerebral blood metrics are determined based on light reflectance data obtained using one or more light emitters and one or more light detectors disposed on a first head-worn device worn by users in the group of users, and wherein the trained machine learning model is provided to a computing device of a second head-worn device on which one or more light emitters and one or more light detectors are disposed.

Embodiment 28: The method of any one of embodiments 26 or 27, wherein the ground truth stroke risk is obtained based on questionnaire data.

Embodiment 29: The method of any one of embodiments 26-28, wherein the ground truth stroke risk is obtained based on longitudinal stroke occurrence data for users of the group of users.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A method of determining stroke risk, the method comprising:
causing, using one or more light sources disposed on a headset worn by a user, light to be emitted into the head of the user;
obtaining, using one or more light detectors disposed on the headset, information indicative of light reflected from one more structures within the head of the user, wherein a portion of the obtained information spans a time period during which the user was holding their breath;
based on the obtained information, determining one or more cerebral blood metrics;
providing a representation of the one or more cerebral blood metrics as input to a trained machine learning model; and
determining a likelihood the user will experience a stroke over a predetermined future time period based on an output of the trained machine learning model.

2. The method of claim 1, further comprising:
determining an updated likelihood the user will experience a stroke based on updated cerebral blood metrics; and
determining a change between the likelihood and the updated likelihood.

3. The method of claim 2, further comprising providing at least one recommendation based on the change between the likelihood and the updated likelihood.

4. The method of claim 1, wherein the one or more light sources comprise at least two light sources, each configured to emit light in a different wavelength.

5. The method of claim 4, wherein the at least two light sources comprise a laser and a light emitting diode packaged together.

6. The method of claim 4, wherein the at least two light sources comprise a laser configured to emit light in an infrared wavelength range.

7. The method of claim 4, wherein the at least two light sources comprise a light emitting diode configured to emit light in a near-infrared wavelength range.

8. The method of claim 1, wherein the one or more light detectors comprise at least two detectors of different types.

9. The method of claim 8, wherein the at least two detectors of different types comprise at least one camera.

10. The method of claim 9, wherein the at least one camera is configured to capture images comprising a speckle pattern.

11. The method of claim 1, wherein the one or more cerebral blood metrics comprise at least one of: cerebral blood flow, cerebral blood oxygenation, or cerebral blood volume.

12. The method of claim 11, wherein the cerebral blood flow is determined based on a decorrelation time associated with a series of speckle patterns obtained from a series of images captured by at least one camera included in the one or more light detectors.

13. The method of claim 11, wherein the representation of the obtained information comprises a ratio of change in cerebral blood flow during the time period the user was holding their breath to a baseline period to a change in cerebral blood volume during the time period the user was holding their breath to a baseline period.

14. The method of claim 1, wherein providing the representation of the one or more cerebral blood metrics as input comprises providing raw traces of the one or more cerebral blood metrics as a function of time to the trained machine learning model, and wherein the trained machine learning model is a deep neural network (DNN).

15. The method of claim 1, wherein providing the representation of the one or more cerebral blood metrics as input comprises providing features of the one or more cerebral blood metrics as an input to trained machine learning model.

16. A system for determining stroke risk, the system comprising:
a headband configured to encircle a head of a wearer of a headset;
a plurality of light sources attached to the headband;
a plurality of light detectors attached to the headband; and
one or more processors configured to:
cause, using the one or more light sources, light to be emitted into the head of the wearer;
obtain, using the one or more light detectors, information indicative of light reflected from one more structures within the head of the wearer, wherein a portion of the obtained information spans a time period during which the wearer was holding their breath;
based on the obtained information, determine one or more cerebral blood metrics;
determine a likelihood the wearer will experience a stroke over a predetermined future time period based on the one or more cerebral blood metrics.

17. The system of claim 16, wherein the plurality of light sources comprise a plurality of light emission packages, each light emission package comprising at least two light sources configured to emit light in different wavelengths.

18. The system of claim 17, wherein the at least two light sources comprise a laser configured to emit light in an infrared wavelength range, and a light emitting diode (LED) configured to emit light in a near infrared wavelength range.

19. The system of claim 16, wherein the plurality of light detectors comprise a plurality of light detection packages, each light detection package comprising at least two light detectors.

20. The system of claim 19, wherein a light detector of the at least two light detectors of a light detection package comprises a camera.

21. The system of claim 20, wherein the obtained information comprises a speckle pattern obtained using images captured by the camera.

22. The system of claim 21, wherein the one or more cerebral blood metrics comprise a cerebral blood flow determined based on the speckle pattern.

23. The system of claim 16, wherein a distance between a light source of the one or more light sources and a light detector of the one or more light detectors is adjustable by changing a position of the light source and/or a light detector on the headband.

24. A method of determining stroke risk, the method comprising:
causing, using one or more light sources disposed on a headset worn by a user, light to be emitted into the head of the user;
obtaining, using one or more light detectors disposed on the headset, information indicative of light reflected from one more structures within the head of the user, wherein a portion of the obtained information spans a time period during which the user was holding their breath;
based on the obtained information, determining a cerebral blood flow as a function of time and a cerebral blood volume as a function of time, wherein the cerebral blood flow and the cerebral blood volume include the time period during which the user was holding their breath and a baseline time period before the user was holding their breath; and
determining a likelihood the user will experience a stroke over a predetermined future time period based on the cerebral blood flow and the cerebral blood volume.

25. The method of claim 24, wherein the likelihood the user will experience the stroke is based on a ratio of a percentage change of cerebral blood flow from a peak after the user began holding their breath to a baseline cerebral blood flow during the baseline time period to a percentage change of cerebral blood volume from a peak after the user began holding their breath to a baseline cerebral blood volume during the baseline time period.

26. A method of training a machine learning model to predict stroke risk, the method comprising:
obtaining training data, the training data comprising, for a group of users, representations of cerebral blood metrics, wherein for each training sample, a portion of the obtained data spans a time period during which the user was holding their breath, and wherein each training sample includes a corresponding ground truth stroke risk for the user;
providing the training data to a machine learning model, wherein the machine learning model takes, as input, the representations of the cerebral blood metrics and generates, as an output, a prediction of stroke risk; and
updating the machine learning model based on differences between the ground truth stroke risk and the predicted stroke risk to generate a trained machine learning model configured to predict stroke risk.

27. The method of claim 26, wherein the representations of cerebral blood metrics are determined based on light reflectance data obtained using one or more light emitters and one or more light detectors disposed on a first head-worn device worn by users in the group of users, and wherein the trained machine learning model is provided to a computing device of a second head-worn device on which one or more light emitters and one or more light detectors are disposed.

28. The method of claim 26, wherein the ground truth stroke risk is obtained based on questionnaire data.

29. The method of claim 26, wherein the ground truth stroke risk is obtained based on longitudinal stroke occurrence data for users of the group of users.
